(19) United States
(12) Patent Application Publication
(10) Pub. No.: US 2020/0078047 A1
(43) Pub. Date: Mar. 12, 2020

(54) VARIABLE STIFFNESS NEEDLE

(71) Applicant: SPIRATION, INC., d/b/a OLYMPUS RESPIRATORY AMERICA, Redmond, WA (US)

(72) Inventors: Peter Lambe, Seattle, WA (US); Nathan Dale, Redmond, WA (US)

(21) Appl. No.: 16/493,584

(22) PCT Filed: Mar. 31, 2017
(86) PCT No.: PCT/US2017/025449

§ 371 Date: Sep. 12, 2019

(51) Int. Cl. A61B 17/34; A61B 10/04; A61B 1/267

(52) U.S. Cl. CPC A61B 17/3417; A61B 10/04; A61M 25/0054; A61B 2010/045; A61B 1/2676

(57) ABSTRACT

A needle comprising: (a) a walled lumen having: (i) a proximate end; (ii) a distal end; and (iii) a plurality of zones located between the proximal end and the distal end, the plurality of zones comprising: (1) a first zone with a first stiffness value; (2) a second zone connected to the first zone, the second zone having a second stiffness value; (3) a third zone connected to the second zone, the third zone having a third stiffness value; and (4) a fourth zone connected to the third zone, the fourth zone having a fourth stiffness value; wherein some of the plurality of zones include one or more cut patterns in the walled lumen.

### FIELD

The present teachings generally relate to a biopsy needle and specifically to a biopsy needle including a plural of zones with different stiffness values that allow the needle to curve in a predetermined direction.

### BACKGROUND

The present teachings are predicated upon providing a needle that is flexible once extended out of a bronchoscope so that a sample can be obtained during a biopsy for making an accurate assessment. Current systems include a needle that extends out of a bronchoscope into or proximate to tissue to assist in retaining the sample within the needle. The needles are bendable when extended out of the bronchoscope, however, the needles may stop bending or compressed by a force is applied the needle when the needle is proximate a site of interest. The needles when pushed against a structure may lose flexibility so that the needles cease to bend. The flexible regions of the needle may compress when pressed against a structure such that an internal structure within the needle cannot extend out of the needle or the needle cannot be used to take a sample. Examples of needles may be found in Patent Application Publication No. 2013/0102890; 2013/0225997; 2014/0276051; and 2015/0351833; all of which are incorporated by reference herein in their entirety for all purposes.

It would be attractive to have a needle that is flexible but sufficiently rigid to be pressed against a structure without losing flexibility or being able to sample. What is needed is a device with multiple different flexing zones that allow one zone to flex while allowing the flexing region for sampling to retain its shape so that sampling can be performed. It would be attractive to have a stiffer flexing zone at a distal end of the needle with a bending region located proximal of the stiffer flexing zone. What is needed is a device that is flexible while being rigid to take a tissue sample and be pushed through a bronchoscope.

### SUMMARY

The present teachings meet one or more (if not all) of the present needs by providing: a needle comprising: (a) a walled lumen having: (i) a proximate end; (ii) a distal end; and (iii) a plurality of zones located between the proximal end and the distal end, the plurality of zones comprising: (1) a first zone with a first stiffness value; (2) a second zone connected to the first zone, the second zone having a second stiffness value; (3) a third zone connected to the second zone, the third zone having a third stiffness value; and (4) a fourth zone connected to the third zone, the fourth zone having a fourth stiffness value; wherein some of the plurality of zones include one or more cut patterns in the walled lumen.

The teachings herein surprisingly solve one or more of these problems by providing a needle that is flexible but sufficiently rigid to be pressed against a structure without losing flexibility or being able to sample. The present teachings provide a device with multiple different flexing zones that allow one zone to flex while allowing the flexing region for sampling to retain its shape so that sampling can be performed. The present teachings provide a stiffer flexing zone at a distal end of the needle with a bending region located proximal of the stiffer flexing zone. The present teachings provide a device that is flexible while being rigid to take a tissue sample and be pushed through a bronchoscope.

### BRIEF DESCRIPTION OF THE DRAWINGS

FIG. 1 is a side plan view of a needle;

FIG. 2 is a top plan view of the needle of FIG. 1;

FIG. 3 is a close up view of area B of the needle of FIG. 2;

FIG. 4 is a side view of area A of the needle of FIG. 1;

FIG. 5 is a perspective view of the distal end of the needle of FIG. 1;

FIG. 6 is a close-up view of a double helix spiral cut pattern;

FIG. 7 is a close-up view of a dog bone cut pattern;

FIG. 8 is a close up view of a single helix spiral cut pattern;

FIG. 9 is a bottom plan view of a needle;

FIG. 10 is a top plan view of the needle of FIG. 9;

FIG. 11 is a side plan view of the needle of FIG. 9;

FIG. 12 is a close-up view of the area B of FIG. 9;

FIG. 13 is a close-up view of the area D of FIG. 10;

FIG. 14 is a close-up view of the area C of FIG. 11;

FIG. 15 is a cross sectional view of the needle of FIG. 9 cut along line A-A;

FIG. 16 illustrates a close-up view of a fifth zone of FIG. 15;

FIG. 17 illustrates a close-up view of a fourth zone of FIG. 15;

FIG. 18 illustrates a close-up view of a first zone of FIG. 15;

FIG. 19 illustrates a close-up view of a second zone of FIG. 15; and

FIG. 20 illustrates a bronchoscope guiding a needle into a patient.

### DETAILED DESCRIPTION

The explanations and illustrations presented herein are intended to acquaint others skilled in the art with the invention, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The needle of the teachings herein may be inserted into or through a larger device. The larger device may function to perform multiple different objectives during a procedure, one of which may be to take a biopsy sample, or gather a tissue sample. The device may be an endoscope, a bronchoscope, or both. Preferably, the device is a bronchoscope that extends into a patient's lungs. The device may assist in taking a tissue sample from an airway, a lung, or both. The device may be a carrier for the biopsy device and needle. Preferably, the device may include a camera or some other device for providing an image. The device may include one or more channels that extend therethrough for accessing a feature at a distal end of the device. The channels may extend from a proximal end or proximal end region to a distal end or distal end region of the device. The channels may receive one or more parts of a needle and direct a distal end of a needle to a location of interest.

The needle may function to extend into a site of interest, a feature of interest, or both and assist in removing a sample. The needle may function to be axially extended and resist deformation when contacted by a feature, tissue, or both. The needle may axially extend and then flex as the needle extends out of the device (e.g., bronchoscope). The needle may extend from a sheath, a deployment device, a delivery device, or a combination thereof. The needle may be free of contact with a sheath, inclusion in a sheath, or both. The needle may include a biopsy device that extends from a needle end (e.g., opening) of the needle. The needle may be about 25 gauge or larger, about 20 gauge or larger, about 18 gauge or larger, about 16 gauge or larger, 14 gauge or larger, about 12 gauge or larger, or even about 10 gauge or larger. The needle may be about 4 gauge or smaller, about 6 gauge or smaller, or even about 8 gauge or smaller. The needle may have a length of about 50 mm or more, about 65 mm or more, about 75 mm or more, or about 85 mm or more. The needle may have a length of about 300 mm or less, about 200 mm or less, about 150 mm or less, or about 100 mm or less. The needle may be made of one or more pieces, two or more pieces, or even three or more pieces. Preferably, the needle is a single piece that includes a plurality of different zones. Each of the zones may be part of the needle. The needle may be made of metal. The needle may be made of a metal that is elastically deformable, plastically deformable, or both. Preferably, the needle may be made of stainless steel. More preferably, the needle is made of surgical stainless steel. The needle may be made of one solid piece and then cut with a plurality of cut patterns. The needle may have a walled lumen with an open area, an open center, a hollow space that extends through the longitudinal axis of the needle (e.g., from a distal end to a proximal end of the needle).

The proximal end of the needle may function to be an end of the needle that is manipulated by a user, contacts a needle that is manipulated by a user, remain outside of the endoscope, or a combination thereof. The proximal end may function to receive a tube or probe that assists in extending the needle through the device (e.g., bronchoscope). The proximal end may be include one or more steps. The proximal end may include a counter bore.

The one or more steps may prevent a pushing device from extending further into the needle. The one or more steps may function to allow the needle to be pushed through the device. The one or more steps may be a counter bore (e.g., a bore with a larger diameter than the diameter of the opening. The one or more steps may vary the wall thickness of the lumen wall. The one or more steps may extend inward so that a pushing device contacts the steps and moves the needle. The proximal end may be located opposite a distal end.

The distal end may be an end of the needle that extends out of the device. The distal end may contact tissue. The distal end may receive a tissue sample. The distal end may be a needle end of the needle. The needle end may have a point, a tip, a sharp edge, opening, or a combination thereof. The needle end may include an opening that allows for tissue to extend into the needle.

The opening may function to receive tissue. The opening may function to allow a sampling device to extend from the distal end or needle end into tissue to take a sample. The opening may be sufficiently large to receive a tissue sample, have a sampling device extend from the needle, or both. The opening may extend from distal end to the proximal end of the needle. The opening may extend through the longitudinal axis of the needle. The opening may be offset form the longitudinal axis of the needle. The opening may be located on a curved portion of the needle and be located from a longitudinal axis of the needle. The opening may move as the needle flexes. The opening may include one or more tapers.

The one or more tapers may function to create a sharp edge. The taper may function to create a sharp edge. The taper may extend inward toward the opening, outward from the opening, or both. One taper may extend towards an opening and one taper may extend away from the opening. The taper may extend away from the opening at an angle.

The taper may extend away from a plane of the opening at an angle. The angle may be about 10 degree or more, about 20 degrees or more, about 25 degrees or more, about 30 degrees or more. The angle may be about 45 degrees or less, about 40 degrees or less, or about 35 degrees or less (e.g., about 30 degrees ±1 degree). The taper may assist in moving the needle into tissue so that a sample may be taken. The taper may be located around an opening and the taper may be formed within the walled lumen (e.g., the opening may be a recess that extends through the walled lumen).

The walled lumen may function to form the body of the needle. The walled lumen may a cylindrical tube of the needle. The walled lumen may be the lumen wall that has a generally circular cross-section. The walled lumen be made of metal. The walled lumen may be an outer wall of the needle. The walled lumen may be made of metal. The walled lumen may be made of stainless steel. The walled lumen may be a singular lumen wall. The lumen wall may have a generally circular cross-section. The lumen wall may form the entirety of the needle. The lumen wall may be tapered at the needle end to form a needle. The lumen wall may include a plurality of zones.

The plurality of zones may each function to have a stiffness value. The plurality of zones may each of different stiffness values. Some of the plurality of zones may different stiffness values and some of the plurality of zones may have the same stiffness values. The plurality of zones may function to vary the flexibility of the needle. The plurality of zones may function to vary the shape of the needle, vary the flexibility of the needle, or both. The plurality of zones may extend between the distal end and the proximal end. The plurality of zones may be located end to end. The plurality of zones may each include a cut pattern. Some of the plurality of zones may be free of a cut pattern. The plurality of zones may be a first zone, second zone, third zone, fourth zone, fifth zone, sixth zone, or a combination thereof. Each of the zones may include a stiffness value. The first zone may include a first stiffness value. The second zone may include a second stiffness value. The third zone may include a third stiffness value. The fourth zone may include a fourth stiffness value. The fifth zone may include a fifth stiffness value. The sixth zone may include a sixth stiffness value. The first zone may be located at the distal end and subsequent zones may extend towards the proximal end. The length of each zone may vary. All of the zones may have a same length. Each zone may be about 1 mm or more, about 2 mm or more, about 3 mm or more, about 4 mm or more, about 5 mm or more, about 10 mm or more or about 15 mm or more. Some of the zones may be about 20 mm or more, about 25 mm or more, or even about 30 mm or more. Some of the zones may be about 100 mm or less, about 75 mm or less, about 50 mm or less, or about 40 mm or less. Some of the zones include a cut pattern.

The cut pattern functions to allow flexibility in the zones of the needle. The cut patterns may extend a full length of a zone. The cut patterns may extend a partial length of a zone. Some of the zones may be free of cut patterns. The cut patterns may change from zone to zone. Each of the cut patterns may have a length.

The length of the cut pattern may be about 1 mm or more, about 2 mm or more, or about 2.5 mm or more from a center of one cut within a cut pattern to a center of a second cut within a cut pattern. For example, one dog bone cut may have a center that may be located about 2.54 mm from a center of another dog bone cut. In another example, one spiral cut may be located about 2.54 mm from another spiral cut. The length of the cut pattern may be referred to as pitch (e.g., spiral length). The pitch of the spiral cuts may be about 1 mm or more, about 2 mm or more, or about 2.5 mm or more. The pitch may be about 10 mm or less, about 7 mm or less, or about 5 mm or less. The pitch may be the distance in length that the cut travels in one revolution around the needle. The cuts may have a thickness through the needle. The thickness of the cut (e.g., scribe kerf) may be about 0.1 mm or more, about 0.2 mm or more, about 0.3 mm or more. The thickness of the cut may be about 1 mm or less, about 0.75 mm or less, or about 0.5 mm or less. The thickness of the cut may determine the gap as discussed herein. The cut pattern may be one or more location patterns (e.g., grooves) in the needle (e.g., lumen wall). The location patterns may be a spiral cut. The location patterns may be concentric non joining rings. The cut pattern may be partial cut into the needle. The partial cuts may have a groove scribe length (e.g., length of the location patterns plus uncut lumen wall between two location patterns). The groove scribe length (e.g., pitch) may be about 0.01 mm or more, about 0.05 mm or more, or preferably about 0.1 mm or more. The groove scribe length (e.g., pitch) may be about 1 mm or less, about 0.5 mm or less, about 0.3 mm or less, about 0.25 mm or less, about 0.2 mm or less, or about 0.15 mm or less (e.g. preferably about 0.1 mm). The location patterns (e.g., grooves) may have a depth. The depth of the location patterns (e.g., grooves) may be less than the thickness of the wall. The groove depth may about 0.005 mm or more, about 0.01 mm or more, or about 0.02 mm or more. The groove depth may be about 2 mm or less, about 1 mm or less, or about 0.5 mm or less. The location patterns (e.g., grooves) may have a length (e.g., kerf) of about 0.005 mm or more, about 0.01 mm or more, or about 0.02 mm or more (e.g., preferably about 0.025 mm). The groove length may be about 2 mm or less, about 1 mm or less, or about 0.5 mm or less. Some of the cut patterns may extend fully through the lumen wall and some may extend partially through the lumen wall. The cut patterns may be a location pattern, single helix spiral cut, double helix spiral cut, dog bone cut, or a combination thereof.

The single helix spiral cut (SHSS) may function to add flexibility to the needle. The SHSS may allow for some flexibility of the needle. The SHSS may allow the needle to bend in virtually any direction. The SHSS may include one spiral cut that extends around the needle. The SHSS may have 1 or more, 2 or more, 3 or more, 4 or more, or even 5 or more revolutions around the needle. The SHSS may have 10 or less, 8 or less, or 6 or less revolutions around the needle. The SHSS may be a single spiral cut that continuously extends around the needle in one endless spiral cut. Each of the spirals may run around the needle in a parallel fashion. The SHSS may be in a first zone, second zone, third zone, fourth zone, or fifth zone. The SHSS preferably is in a second zone. The SHSS may have a stiffness value. The stiffness value of the SHSS have be a stiffness value of the zone in which the cut is located. For example, if SHSS is located in the second zone then the SHSS has a second stiffness value. The SHSS may have a lower stiffness value than the location patterns (e.g., grooves), the needle without cuts, or both. The SHSS may have a higher stiffness value in at least one direction than the double helix spiral cut and the dog bone cut. The SHSS may be located proximate to a double helix spiral cut, a dog bone cut, location patterns (e.g., grooves), or a combination thereof.

The double helix spiral cut (DHS) may function to allow for a flexibility in a plurality of directions. The DHS may add flexibility to the needle. The DHS may include two spiral cuts that extend around the needle. The two spiral cuts extend around the needle in two parallel spiral cuts. Thus, the DHS may have a first spiral cut and a second spiral cut. The first spiral cut and the second spiral cut may be parallel. The spirals may be located apart from each other a distance discussed herein. The spirals of the DHS may be located closer together than the spirals of the SHSS. The DHS may have 1 or more, 2 or more, 3 or more, 4 or more, or even 5 or more revolutions around the needle. The DHS may have 10 or less, 8 or less, or 6 or less revolutions around the needle. The DHS may be located in the first zone, second zone, third zone, fourth zone, or fifth zone. The DHS preferably is in a fourth zone. The DHS may have a stiffness value. The stiffness value of the DHS have be a stiffness value of the zone in which the cut is located. For example, if DHS is located in the fourth zone then the DHS has a fourth stiffness value. The DHS may have a lower stiffness value than the location patterns (e.g., grooves or dimples), the needle without cuts, SHSS, or a combination thereof. The DHS may have a higher stiffness value in at least one direction than the dog bone cut. The SHSS may be located proximate to a double helix spiral cut, a dog bone cut, location patterns (e.g., grooves), or a combination thereof.

The one or more location patterns function to assist a user in locating the needle while the needle is located within a user. The location patterns may be detectable by MRI, ultrasound, or both. Preferably, the location patterns are detectable by ultrasound in real time. The location patterns may be located in a first zone, second zone, third, zone, fourth zone, fifth zone, or a combination thereof. Preferably, the location patterns are located within the first zone, at the distal end, or both. The location patterns may have a stiffness that is greater than the SHSS, the DHS, the dog bone cuts, or a combination thereof. The location patterns may have a stiffness that is substantially equal to the stiffness of an uncut needle. The location patterns may have a stiffness that less than the uncut needle. The locations patterns may not be flexible. The location patterns may not allow the needle to flex. The location patterns may be a plurality of grooves that extend around the needle. The grooves may be a cut that extends partially around the needle. The grooves may have a beginning and an end that connect. The grooves may spiral around the needle. The location patterns may be dimples. The dimples may be generally circular cuts into the lumen wall. The dimples may be randomly located in the lumen wall. The dimples may have patterns that extend around the lumen wall. The dimples may be located throughout a first zone and may stop before a SHSS, DHS, or dog bone cut.

The dog bone cut (DBC) may function to permit single plane flexibility of the needle. The DBC may allow the needle to only flex in a single direction. The DBC assist in bending the needle in a predetermined direction. The DBC may be located in a first zone, second zone, third zone, fourth zone, fifth zone, or a combination thereof. Preferably, the DBC is located within the third zone. The DBC may be located on a first side of the needle, a second side of the needle, or two sides of the needle. The needle may include about 5 pairs DBCs or more, about 10 pairs DBCs or more, about 15 pairs DBC or more, or about 19 pairs DBCs or more along a length of the needle. The needle may have about 30 pairs of DBCs or less or about 25 DBCs or less. The DBC may have a first half that is located about 180 degrees from a second half. The DBC may have a stiffness value in the predetermined direction (i.e., direction of flexing) that is less than the stiffness value of the DHS, SHSS, the locations patterns, an uncut tube, or a combination thereof. The DBC may include one or more flexible regions.

The one or more flexible regions may function to permit the needle to flex in a predetermined direction. The one or more flexible regions may function to permit the needle to flex so that the needle has a curve of about 2 mm or more, about 5 mm or more, about 8 mm or more in diameter. The one or more flexible regions may function to permit the needle to flex so that the needle has a curve of about 20 mm or less, about 15 mm or less, about 10 mm or less in diameter. The flexible regions may permit the needle to only bend in a single direction. Each DBC may include a flexible region. The flexible regions may have one or more pieces. The flexible regions may have two or more pieces. Preferably, each flexible region includes a first piece and a second piece that are movable to each other.

The first piece and the second piece function to movable relative to each other to allow the needle to flex in a predetermined direction. The first piece and the second piece may be connected by a connection region (i.e., a region of the needle that is uncut). The first piece, the second piece, or both may be generally “U” shaped, generally “C” shaped, or both. The first piece may include a portion that extends into the second piece or vice versa. The first piece may extend around 1 or more sides, 2 or more sides, or even 3 or more sides of the first piece or vice versa. The first piece and the second piece may be complementary in shape. The first piece and the second piece may be mirror images of each other. The needle may include a first piece and a second piece that are complementary on a first side of a needle and a first piece and a second piece that are mirror images on a second side of a needle. The first piece and the second piece may be separated from the connection region by a cut. The first piece and the second piece may be separated by a cut, a gap, or both.

The gap may function to allow the first piece to move relative to the second piece. The gap may increase during flexing. The gap may decrease during flexing. The gap may be sized to limit the amount of flexing of the needle. The gap may set the amount of flexing the needle does. For example, during flexing the gap may decrease in size so that the first part and the second part come into contact and further flexing is eliminated. The gap may be measured from the closest distance between the first part and the second part. The gap may be a cut between the first piece and the second piece.

The cut may form the first piece and the second piece. The cut may function to permit movement of the first piece relative to the second piece. The cut may weaken the needle in the flexible regions so that the needle flexes. The cut may include one or more relief zones that prevent the cut from changing sizes during flexing.

The one or more relief zones function to maintain the size and shape of the cut during flexing. The one or more relief zones may prevent the cut from increasing in size. The one or more relief zones may be a terminal end of the cut. The one or more relief zones may be circular, oval, square, triangular, rectangular, or a combination thereof. The one or more relief zones may prevent the first part, the second part or both to move relative to each other. The one or more relief zones may be located in a high stress concentration area. The one or more relief zones may extend partially into the connection region and partially into the first part or the second part.

The one or more connection regions may be an uncut region of the needle. The one or more connection regions may be extend between a first DBC and a second DBC. The one or more connection regions may separate the first DBC and the second DBC. The one or more connection regions may be generally straight. The needle may include two connection regions at each DBC and the two connection regions may be located across from each other (e.g. a center at about 180 degrees apart). The connection regions may include two generally linear edges.

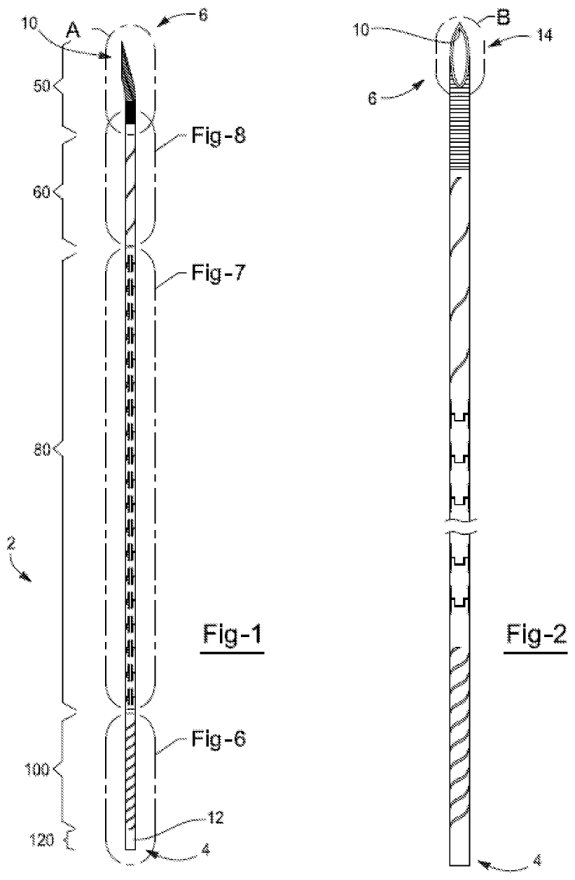

FIG. 1 illustrated a side plan view of a needle 2. The needle 2 is a walled lumen 12 having a proximal end 4 and a distal end 6. A first zone 50, second zone 60, third zone 80, fourth zone 100, and fifth zone 120 extend between the proximal end 4 and the distal end 6. The distal end of the needle 2 includes a needle end 10.

FIG. 2 illustrates a top plan view of the needle 2 of FIG. 1. The needle 2 including a proximal end 4 and a distal end 6 with an opening 14 and a needle end 10 at the distal end 6.

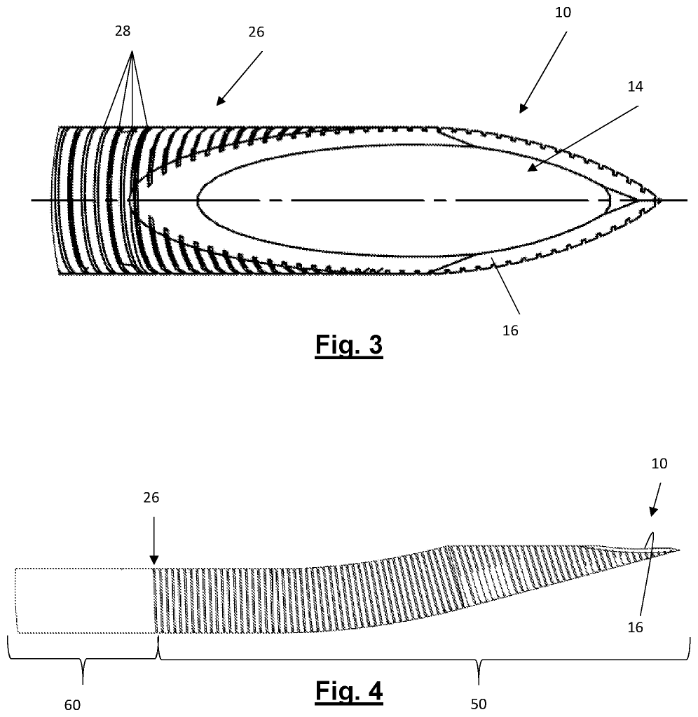

FIG. 3 is a close up view of the needle end 10 and the opening 14 in area B of FIG. 2. A lumen wall 16 extends around the opening 14. The lumen wall 16 includes a plurality of locations patterns 26 that are shown as grooves 28 extending into the lumen wall 16.

FIG. 4 illustrates a side view of a first zone 40 and second zone 60 in area A of FIG. 1. The first zone 40 shown the needle end 10 of the lumen wall 16 extending at an angle. The first zone 40 ends where the location patterns 26 end and the second zone 60 begins after the location patterns.

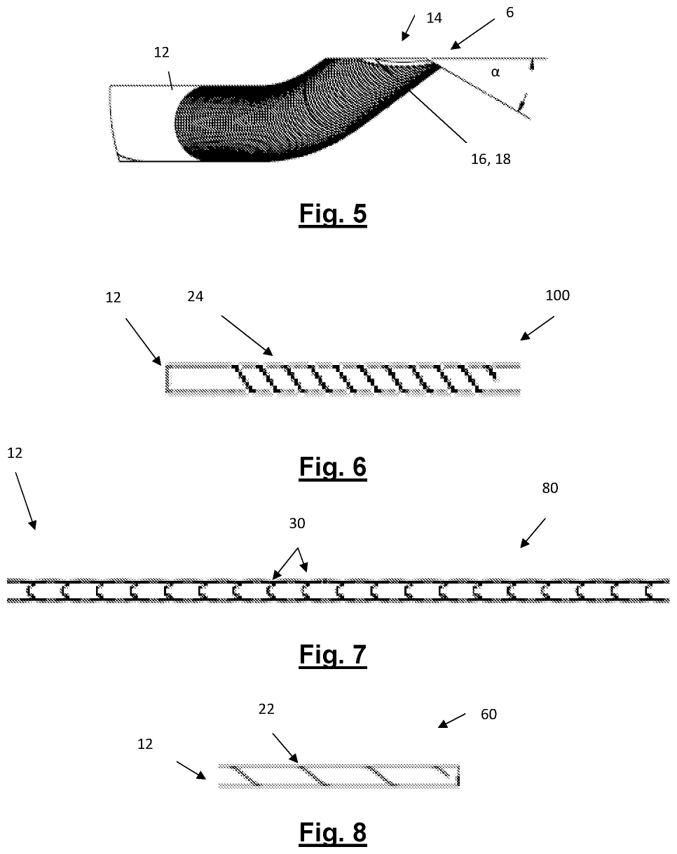

FIG. 5 illustrates a perspective end view looking from the distal end 6 towards the proximal end. The walled lumen 12 has a lumen wall 16 and an opening formed within the lumen 12. The lumen wall 16 has a taper 18 away from the opening extending at an angle (α).

FIG. 6 illustrates a close-up view of a cut pattern 20 in the walled lumen 12 of the fourth zone 100. The cut pattern 20 as shown is a double helix spiral cut 24.

FIG. 7 illustrates a close-up view of a cut pattern 20 in the walled lumen 12 of the third zone 80. The cut pattern 20 as shown is a dog bone cut 30.

FIG. 8 illustrates a close-up view of a cut pattern 20 in the walled lumen 12 of the second zone 60. The cut pattern 20 as shown is a single helix spiral cut 22

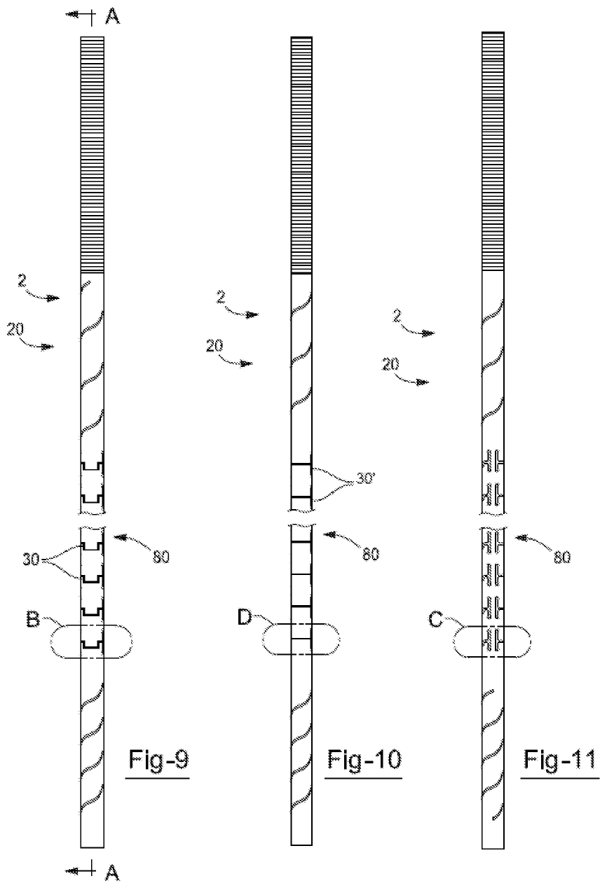

FIG. 9 illustrates a bottom plan view of a needle 2 with a third zone 80 including a cut pattern 20 that is a dog bone cut 30.

FIG. 10 illustrates a top plan view of the needle 2 of FIG. 9 with the cut pattern 20 of the third zone 80 that is located opposite the cut pattern of FIG. 9. The cut pattern 20 is a dog bone cut 30′ that differs from the dog bone of FIG. 9.

FIG. 11 illustrates a side plan view of the needle 2 of FIGS. 9 and 10 so that a portion of the cut patterns 20 of the third zone 80 are each visible.

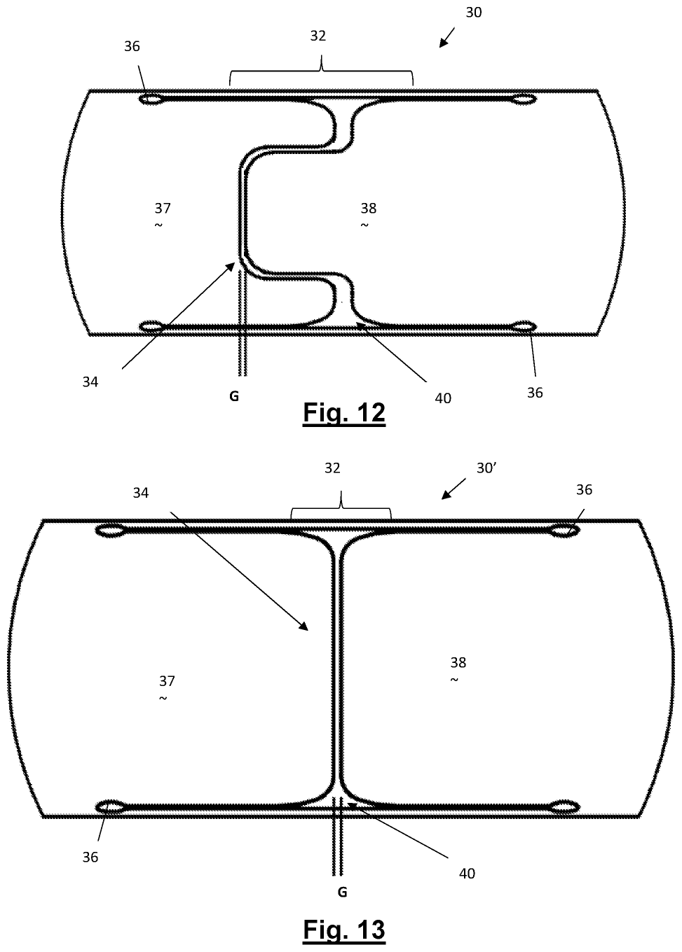

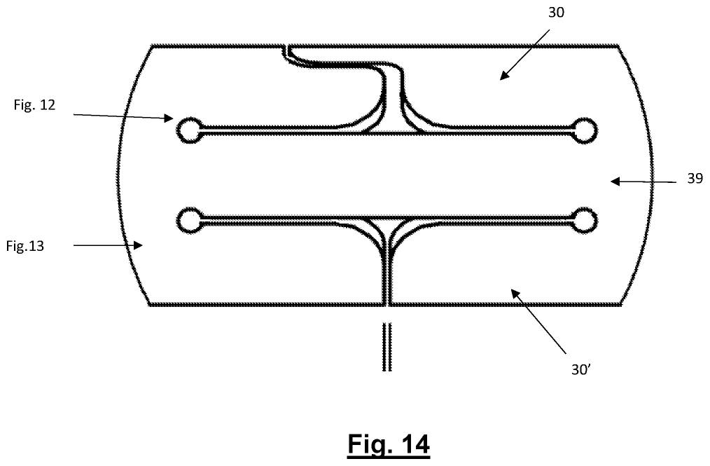

FIG. 12 illustrates a close-up view of area B of FIG. 9. The dog bone pattern 30 includes: a flexible region 32 with a gap 34 (G) separating a first piece 37 and a second piece 38. The first piece 37 and the second piece 38 include a relief zone 36 that form a terminal portion of the cut 40 around the first piece 37 and the second piece 38 respectively. During bending the first piece 37 and the second piece 38 move towards each other so that the gap (G) is reduced and the first piece 37 and the second piece 38 possibly contact each other.

FIG. 13 illustrates a close-up view of area D of FIG. 10. The dog bone cut 30′ pattern includes: a flexible region 32 with a gap 34 (G) separating a first piece 37 and a second piece 38. The first piece 37 and the second piece 38 include a relief zone 36 that form a terminal portion of the cut 40 around the first piece 37 and the second piece 38 respectively. During bending the first piece 37 and the second piece 38 move away from each other so that the gap (G) increases in size.

FIG. 14 illustrates a close-up view of area C of FIG. 11. Area C is a connection region 39 between the dog bone cut 30 of FIG. 12 and the dog bone cut 30′ of FIG. 13. A mirror connection region is located on an opposite side of the needle from the shown connection region 39.

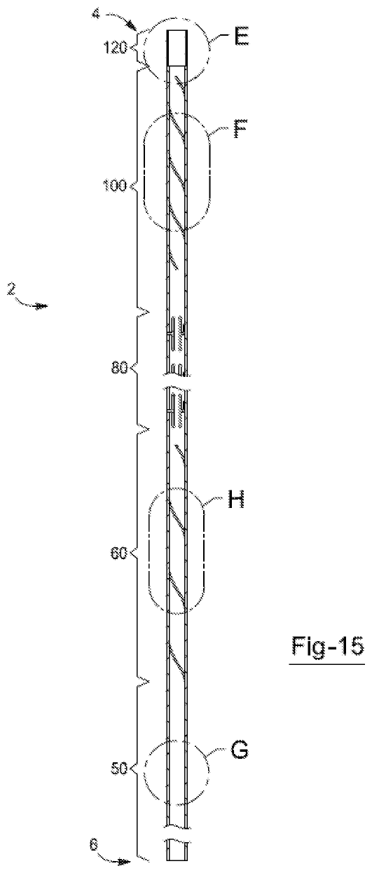

FIG. 15 illustrates a cross-sectional view of the needle 2 cut along lines A-A. The needle 2 has a proximal end 4 and a distal end 6. Beginning at the proximal end 4 and extending to the distal end there is a first zone 50, a second zone 60, third zone 80, fourth zone 100, and fifth zone 120.

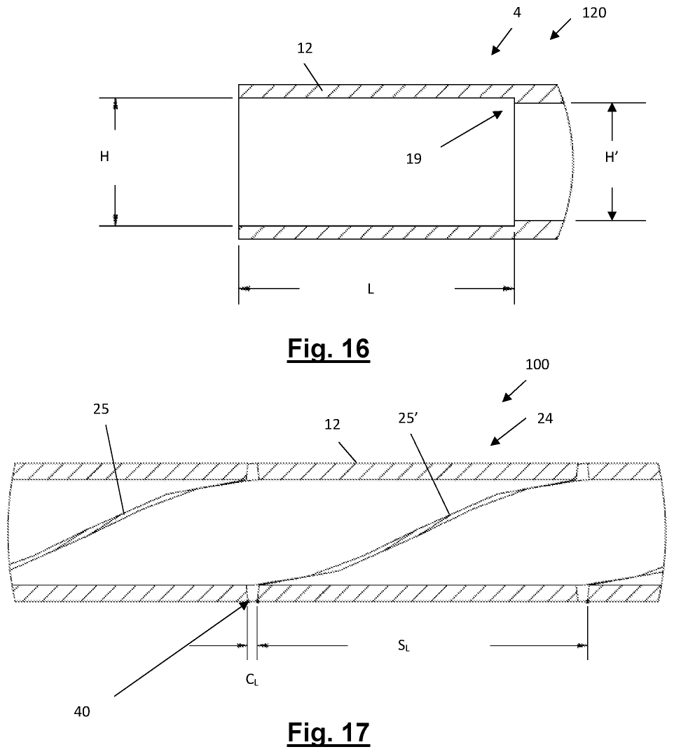

FIG. 16 illustrates a close-up view of the fifth zone 120 at the proximal end 4 of the walled lumen 12 in area E of FIG. 15. The fifth zone 120 is free of a cut pattern. The fifth zone 120 has a lumen 12 including a step 19. The interior height of the lumen 12 changes from (H) to (H′) as the step so that another tube when placed in the lumen 12 can only extend the length (L) and is prevented from extending further by the step 19.

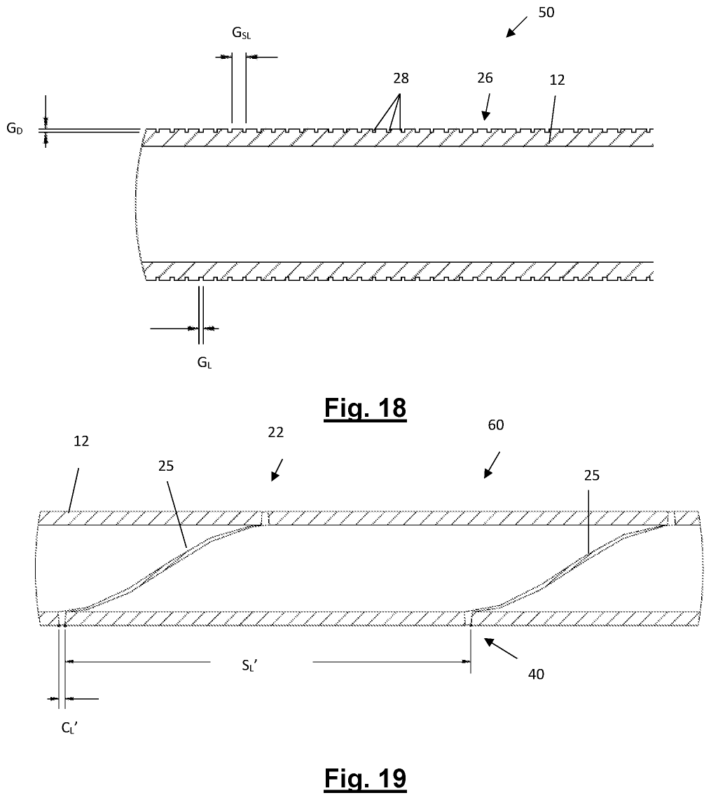

FIG. 17 illustrates a close-up view of the fourth zone 100 in area F of FIG. 15. The fourth zone 100 includes a lumen 12 that has a double helix spiral cut 24. The double helix spiral cut 24 has a first spiral cut 25 and a second spiral cut 25′ that extend parallel to one another around the lumen 12. The spiral cuts 25, 25′ have a thickness through the lumen of (C_{L}). The spiral cuts 25, 25′ are separated by a length (S_{L}) (e.g., pitch).

FIG. 18 illustrates a close-up view of the first zone 50 in area G of FIG. 15. The first zone 50 has a walled lumen 12 having a location pattern 26. The location pattern 26 includes a plurality of grooves 28. The grooves have a groove length (G_{L}), a groove depth (G_{D}) and a groove scribe length (G_{SL}).

FIG. 19 illustrates a close-up view of the second zone 60 in area H of FIG. 15. The second zone has a walled lumen 12 having a single helix spiral cut 22. The single helix spiral cut 22 includes a spiral cut 25 that extends through the lumen 12 at a cut 40. The spiral cut 25 has a length or pitch (S_{L}′) and the cut 40 has a thickness (C_{L}′).

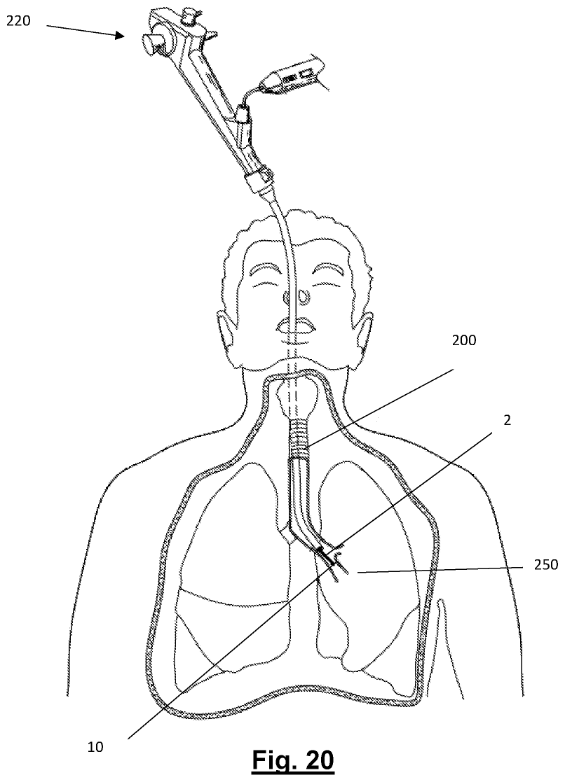

FIG. 20 illustrates a bronchoscope 220 extending into an airway 200. The needle 2 is located inside of the bronchoscope 220 as the needle end 10 extends out of the bronchoscope 220 to sample tissue 250.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of the elements, ingredients, components or steps. By use of the term “may” herein, it is intended that any described attributes that “may” be included are optional.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

2 Needle

4 Proximal end

6 Distal end

10 Needle end

12 Walled lumen

14 opening

16 Lumen wall

18 taper

19 Step

20 Cut pattern

22 Single helix spiral cut

24 Double helix spiral cut

25 Spiral cut

26 Location pattern

28 Grooves

30 Dog bone cut

32 Flexible region

34 Gap

36 Relief zone

37 First piece

38 Second piece

39 Connection region

40 Cut

50 First zone

60 Second zone

80 Third zone

100 Fourth zone

120 Fifth zone

200 Airway

220 Bronchoscope

250 Tissue

S_{L} Spiral length

C_{L} Cut thickness in lumen

G_{L} Groove length

G_{D} Groove Depth

G_{SL} Groove scribe depth

1. A needle comprising:
a. a walled lumen having:
i. a proximal end;
ii. a distal end; and
iii. a plurality of zones located between the proximal end and the distal end, the plurality of zones comprising:
1. a first zone with a first stiffness value;
2. a second zone connected to the first zone, the second zone having a second stiffness value;
3. a third zone connected to the second zone, the third zone having a third stiffness value; and
4. a fourth zone connected to the third zone, the fourth zone having a fourth stiffness value;
wherein some of the plurality of zones include one or more cut patterns in the walled lumen.

2. The needle of claim 1, wherein the needle includes a fifth zone connected to the fourth zone, the fifth zone having a fifth stiffness value.

3. The needle of claim 1, wherein the needle is configured to sample tissue from an airway of a subject.

4. The needle of claim 1, wherein the needle is configured to extend through a bronchoscope into a subject.

5. The needle of claim 1, wherein the one or more cut patterns of the first zone comprises a first spiral scribe cut.

6. The needle of claim 1 wherein the one or more cut patterns of the second zone comprise a second spiral scribe cut.

7. The needle of claim 1 wherein the one or more cut patterns of the third zone comprise a dog-bone cut pattern.

8. The needle of claim 2, wherein the one or more cut patterns of the fourth zone comprise a third spiral cut.

9. The needle of claim 5, wherein the first spiral cut has a scribe pitch ranging from about 0.01 mm to about 0.3 mm, a scribe kerf ranging from about 0.1 mm to about 1 mm, or a combination thereof.

10. The needle of claim 6, wherein the second spiral cut has a scribe pitch ranging from about 1 mm to about 10 mm, a scribe kerf ranging from about 0.005 mm to about 1 mm, or a combination thereof.

11. The needle of claim 8, wherein the third spiral cut has a scribe pitch ranging from about 1 mm to about 10 mm, a scribe kerf ranging from about 0.1 mm to about 1 mm, or a combination thereof.

12. The needle of claim 7, wherein the dog-bone cut pattern is generally “H” shaped.

13. The needle of claim 7, wherein the dog-bone cut pattern has a portion that is generally “U” shaped.

14. The needle of claim 4, wherein the needle has a length that is sufficiently long so that only the distal end of the needle extends from the bronchoscope during tissue sampling.

15. The needle of claim 2, wherein the needle has a length that is sufficiently long so that only the fourth zone of the needle extends from a bronchoscope during tissue sampling.
